(19) United States
(12) Patent Application Publication
(10) Pub. No.: US 2011/0294201 A1
(43) Pub. Date: Dec. 1, 2011

(54) Test device for liquids of the human or animal body

(76) Inventor: Walter Franz, Gemunda (DE)

(21) Appl. No.: 13/138,315

(22) PCT Filed: Feb. 3, 2010
(86) PCT No.: PCT/EP2010/000663

§ 371 Date: Aug. 1, 2011

(51) Int. Cl. C12M 1/34

(52) U.S. Cl. 435/287.6

(57) ABSTRACT

A test device for liquids of the human or animal body, comprises a tube-shaped housing, in which are disposed:
an in-feed area for feeding in a prescribed quantity of a bodily fluid, a reaction area connected downstream of the in-feed area and separated from the same by at least one closed separating wall, and in which the bodily fluid is brought to a chemical reaction with at least one reagent, and an indication area connected downstream of the reaction area and separated from the same by a separating element having a defined through-hole, and in which an indication element is disposed, the bodily fluid being passed through the through-hole after reacting with the reagent. A cutting device is thereby provided by means of which the separating wall can be opened or destroyed.

The invention relates to a test device for liquids of the human or animal body.

In medicine, it has long been known how to analyze liquids of the human or animal body, for example, blood, urine or saliva, in order to acquire deviations from the normal condition of the body at an early stage and thus detect diseases or other changes in condition of the body, for example, pregnancy.

Hereinafter, it shall be assumed by way of example that the bodily fluid to be tested is blood. However, the invention is not limited to this alone. On the contrary, the inventive test device can also be applied to other liquids of the human and animal body and the invention also expressly includes these.

A test device is known with which a prescribed quantity of the blood to be examined is applied to a carrier and is inserted therewith into a reagent with which blood reacts chemically. Either this chemical reaction is manifested by a change that is visible to the user, or the blood is applied to an indicator, for example, a so-called test strip, after reaction with the reagent, where it causes a change in color in dependence on its properties. From the change in color, the user can read whether and, if so, to what extent the examined blood deviates from the normal condition.

The known test device comprises multiple modules or components, which the user must apply one after the other. This procedure is complicated and there is a risk that the user may disregard the sequence of modules and thus invalidate the test or render it useless. Furthermore, there is also a risk that individual components may fall to the floor during use and be soiled, which would also render the test device useless.

The object of this invention is to create a test device of this type that is simple to use and that guarantees a precisely defined test sequence.

This object is inventively solved with a test device with the characteristics of claim 1.

The basic idea of the invention is that all phases of the test be executed in a single, preferably tube-shaped, housing in which various function areas are constituted. The bodily fluids or blood to be examined can be fed in a prescribed quantity into an in-feed area or introduced into the housing. Inside the housing, the blood then passes through the reaction area and finally reaches an indication area with an indication element from which the user can read the test result.

The blood passes from the in-feed area into the reaction area, which is connected downstream of the in-feed area and separated from the same by at least one closed separating wall. At least one reagent is located in the reaction area, with which the blood reacts chemically. In the initial state of the test device, the in-feed area is completely separated from the reaction area by the closed separating wall. In order to transfer the blood from the in-feed area into the reaction area, the invention comprises a cutting device that can be operated by the user to destroy the separating wall, so that the blood flows into the reaction area where it combines with the reagent and reacts chemically.

The indication area is connected downstream of the reaction area, the former being separated from the reaction area by a separating element with a defined through-hole. The indication element, for example a test strip, is disposed in the indication area, to which the blood is fed through the through-hole in a defined manner after reaction with the reagent.

By disposing the function areas within a single tube-shaped housing, it is assured that the blood cannot reach the indication area until it has passed through the reaction area and reacted there with the reagent. Any unintentional change in the sequence of the individual test phases is thus ruled out.

The blood can be introduced into the in-feed area of the tube-shaped housing in any way. In a preferred embodiment of the invention, the in-feed area has a capillary tube into which the blood to be tested can be taken by capillary action. At least one drop of blood is taken from the test person whose blood is to be examined in the usual way, for example, by pricking the finger tip. The needle or pointed object used to do this can be integrated into the test device at a suitable location. However, it is also possible to prick the skin with a separate pointed object. As soon as the drop of blood located on the finger of the test person makes contact with the end of the capillary tube, the capillary action inside the capillary tube causes the blood to be drawn into the capillary tube. In this way, a relatively precisely determined quantity of blood of a few microliters (=10⁻⁹ m³) can be taken.

For the quantity of blood taken up by the capillary tube to flow through the individual areas of the housing of the test device, the blood must flow back out of the capillary tube against the force of the capillary action. This can be achieved, for example, by applying positive pressure at one end of the capillary tube. In a preferred embodiment of the invention, this can be achieved by placing a cap over the capillary tube, which presses the quantity of blood out of the capillary tube. Preferably, the cap has an internal blind hole into which the capillary tube can be inserted without play. When the capillary tube is inserted into the blind hole of the cap, the space between the base of the blind hole and the inserted end of the capillary tube is reduced, which causes an increase in pressure that presses the blood out at the opposite end of the capillary tube.

After the blood has been introduced, it is located in the in-feed area and is separated from the reaction area by the closed separating wall. The user then opens the connection between the in-feed area and the reaction area by opening or destroying the separating wall with the cutting device. In a preferred embodiment of the invention, the cutting device can have a cutting part, which is positioned in the housing so that it can be rotated or moved axially and which has a cutting knife. Because of the ability of the cutting part to move axially, the user can move the cutting knife up against the separating wall to be opened and break through the latter. An ability of the cutting part and of the cutting knife to rotate can assist this opening action.

The cutting part with the cutting knife should preferably execute a precisely defined movement relative to the housing of the test device that is not chosen by the user but is determined by an appropriate guiding device. To this end, a further embodiment of the invention can be provided wherein the movement of the cutting part is controlled by means of a slide, which is constituted in the housing. Preferably, the cutting part has a guide pin that passes through the slide, which is preferably constituted as a guide slot or guide groove, into which it fits tightly. Reliable guidance is assured if two corresponding slide guides are constituted on diametrically opposed sides of the housing. In order to initiate the movement of the cutting part, the cutting part can engage with the cap in a positive connection. The user then turns and/or moves the cap, which is easily accessible to him, and thus also the cutting part along the path of the slide so that a defined movement is achieved.

Alternatively, a thread can also be provided that defines the movement between the cutting part and the housing.

Once the separating wall between the in-feed area and the reaction area has been opened, the blood flows out of the in-feed area into the reaction area and reacts with the reagent located there. In a preferred embodiment of the invention, the reagent is not contained in the reaction area such that it moves freely but is contained in a cartridge that is inserted into the housing and has at least one covering film that can be destroyed using the cutting device. In this way, it is possible to prefabricate the cartridge together with the desired reagent and to insert it into the housing in a filled and closed condition during manufacture of the test device. This makes it possible to deploy different reagents for different applications in the same housing simply by inserting the relevant prefabricated cartridge.

In one possible embodiment of the invention, the cartridge can comprise a tube-shaped housing part that is inserted into the housing of the test device in a tight fit and is closed at both its ends, facing in the axial direction of the housing, with a covering film that, for example, can be applied by sealing. The use of prefabricated cartridges of this design has the advantage that multiple cartridges can be disposed one behind the other in the axial direction of the housing, wherein either the same reagent is contained in each cartridge, which is expedient if a relatively large quantity of reagent is required, or the individual cartridges contain different reagents or antibodies.

The individual cartridges are contiguously sequentially disposed in the axial direction of the tube-shaped housing of the test device and preferably pressed against each other using a clamping element. In this way, the individual cartridges are in a defined position inside the housing. This makes it possible to open the cartridges with the cutting device and allow them to react with the blood not simultaneously but in a defined sequence. To this end, the slide that controls the movement of the cutting part can progress in steps, i.e. exhibit a polygonal progression. In a 1st phase of the action, the cutting part is moved axially until the cutting knife attached to it opens the 1st cartridge so that the blood is mixed with the reagent located in the 1st cartridge and reacts with it. The further cartridges located axially behind the 1st cartridge initially remain intact. If the cutting part is then turned relative to the housing, a 2nd axial movement of the cutting part along the slide is then possible, whereby the cutting knife opens the next cartridge and the blood can also react with the reagent of this 2nd cartridge.

Once the blood in the reaction area has reacted with the reagent or reagents, it flows toward the separating element that separates the reaction area from the following indication area but which has a defined through-hole with very small dimensions, i.e. a diameter of less than 1 mm. The blood that makes contact with the separating element can flow through the through-hole and then make contact with the indication element, for example, a test strip suitable for the application at hand, which can change color depending on the test result.

In order to be able to reliably guide the blood toward the through-hole once it has made contact with the separating element, in a further embodiment of the invention, the separating element can be bowl-shaped and fit tightly inside the housing. Here, the through-hole can preferably be constituted in the base of the separating element. The bowl shape of the separating element ensures that the blood is collected in the latter and then flows through the through-hole constituted in the base and can enter the indication element.

When the separating wall between the in-feed area and the reaction area is destroyed with the cutting device and on opening the covering film of the cartridges, individual pieces of the film can become detached and collect in the bowl-shaped separating element. To prevent such a piece of film from coming to rest in front of the through-hole and unintentionally closing it, the through-hole is preferably surrounded by spacer elements, which do not prevent the blood from flowing into the through-hole but do considerably reduce the danger of blockage of the through-hole by cut-out pieces of film as the film pieces are retained by the spacer elements before they can come to rest directly on the through-hole.

The indication element is preferably constituted as an indicator strip or test strip which is aligned in the longitudinal direction of the tube-shaped housing of the test device and which can be viewed by a user from outside the housing after execution of the test and which is either transparent or at least has a transparent window in the area of the indicator.

In a preferred embodiment of the invention, a retainer for the indication element is constituted on the side of the separating element that faces the indication element. The retainer can be a tube-shaped projection into which the indication element is inserted, preferably with only slight elastic deformation, and clamped. In this way, the indication element is disposed directly at the mouth of the through-hole thus ensuring that all the blood that has been combined with the reagents and flows through the through-hole enters the indication element.

Preferably, the strip-shaped indication element is also contained in a holder at the opposite end facing away from the separating element to ensure reliable positioning of the indication element relative to the housing.

Further details and characteristics of this invention are provided by the following description of an embodiment with reference to the drawings. The illustrations show:

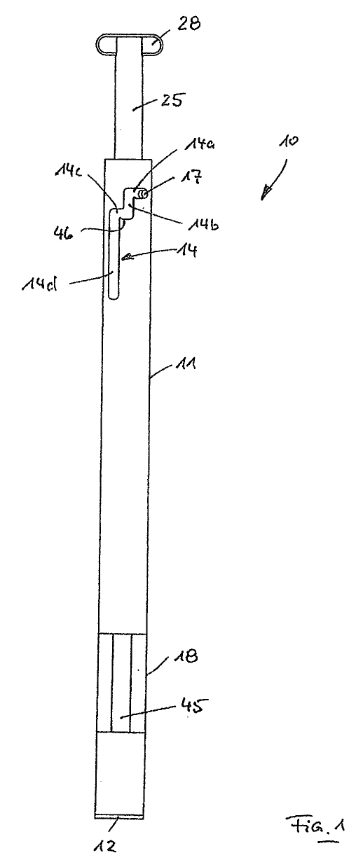

FIG. 1 a side view of an inventive test device,

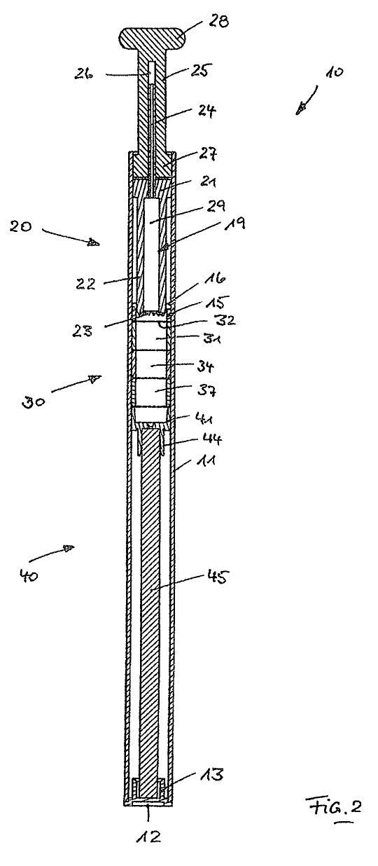

FIG. 2 a longitudinal section through the test device according to FIG. 1,

FIG. 3 an enlarged representation of the in-feed area of the test device,

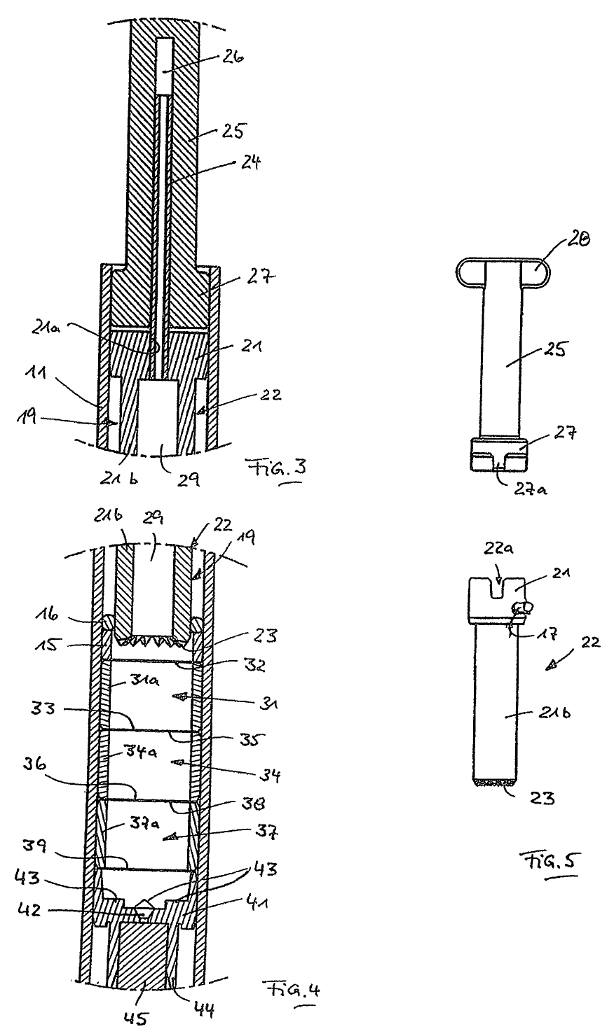

FIG. 4 an enlarged representation of the reaction area of the test device, and

FIG. 5 a side view of the cap and of the cutting part of the test device.

A test device 10 depicted in the figures comprises an elongated, tube-shaped housing 11 that is closed at its lower end by a cap plug 12, as shown in FIGS. 1 and 2. A holder 13 is integrated into the side of the cap plug 12 facing the inside of the housing 11, into which a strip-shaped indication element 45 is inserted and held. The strip-shaped indication element 45 extends along the axial direction of the housing 11 and, at its opposite upper end, slots into a tube-connector-shaped retainer 44 of a bowl-shaped separating element 41. The strip-shaped indication element 45 is positioned securely and immovably by the lower holder 13 in an indication area 40 of the housing 11.

The separating element 41, which separates the lower indication area 40 from a reaction area 30 located above it, has a bowl-shaped cross-section that opens upward and fits tightly in housing 11 in such as way as to ensure sealing. A through-hole 42 extending axially is constituted in the base of the separating element 41 and opens directly onto the end of the strip-shaped indication element. On the upper side of the base of the separating element 41 facing away from the indication element 45, the through bore hole 42 is surrounded by spacer elements 43 (see FIG. 4) that project a small distance upward from the base.

Three cartridges 31, 34, and 37 are disposed directly above the separating element 41, which are disposed one behind the other in the axial direction of the housing 11 and lie one on top of the other. Each cartridge 31, 34, 37 has a tube-shaped housing part 31a, 34a, 37a, whose exterior dimensions correspond to the interior dimensions of the housing 11, and is closed at both the top and bottom end by a covering film 32, 33 or 35, 36 or 38, 39. Each cartridge 31, 34, 37 contains a reagent or some other chemical substance, which is required for the examination of the bodily fluids or of the blood. The cartridges 31, 34, and 37 are prefabricated and are inserted into the housing 11 in the filled and sealed condition so that they fit tightly and make contact in the axial direction with their tube-shaped housing parts 31a, 34a, 37a, as is illustrated, in particular, in FIG. 4. The lower end of the lower cartridge 37 facing indication element 45, lies directly on the upper edge of the bowl-shaped separating element 41. A clamping element 15 in the shape of a clamp sleeve is disposed at the opposite upper end of the upper cartridge 31, the former being pressed against the interior wall of the housing 11 with elastic deformation and thus pressing the three cartridges 31, 34, and 37 against each other in the axial direction and positioning them securely.

The upper film 32 of the upper cartridge 31 forms a separating wall between reaction area 30 of the test device 10 surrounding the cartridges 31, 34 and 37 and an in-feed area 20 lying above it, in which a prescribed amount of the blood to be examined is introduced into the test device 10.

A cutting device 19 which comprises a cutting part 22 is disposed inside the housing above the cartridges 31, 34, 37. The cutting part 22 comprises an upper retaining body 21, which is inserted in the housing 11 with essentially no play, onto whose lower side a tube-shaped projection 21b is molded as an integral part, which holds a cutting knife 23 in the shape of a cutting ring consisting of cutting teeth on its lower end facing the cartridges 31, 34, 37. A ring- or cylinder-shaped sealing element 16 is disposed near the lower end of the tube-shaped projection 21b, and rests on the interior side of the housing 11 and on the external side of the tube-shaped projection 21b and which is supported by the top side of the clamp sleeve 15 in the axial direction.

An axial center hole 21a is constituted in the retaining body 21 of the cutting part 22 into which a capillary tube 24 is inserted and then protrudes upward.

A tube-shaped cap 25 has an inner blind hole 26, with which it can be inserted onto the upwardly protruding section of the capillary tube 24 without play. At its upper end, the cap 25 has a grip piece 28 with which a user can grip the cap 25 and, in particular, rotate and move it axially. A guide part 27 is molded as an integral part onto the lower end of the cap 25 facing away from the grip piece 28, which has an axial projection 27a (see FIG. 5) with which, by means of a retainer 22a of the retaining body 21 of the cutting part 22, it can engage in such a way that a rotational movement applied via the grip piece 28 to the cap 25 is transferred to the cutting part 22.

As FIG. 5 shows, the retaining body 21 of the cutting part 22 has a guide pin 17 that extends radially outward and engages with a control curve in the form of a slit-shaped slide 14 (see FIG. 1) constituted in the housing 11. The slide 14 comprises an upper 1st section 14a that extends in the circumferential direction of the housing 11, a 2nd section 14b adjacent to it that extends in the longitudinal direction of the housing 11, a further adjacent section 14c that extends in the circumferential direction of the housing 11, and a further adjacent 4th section 14d that extends in the longitudinal direction of the housing 11. In the transition area between the 3rd section 14c and the 4th section 14d, an integrally molded nose 46 provides a slight narrowing of the cross-section, which is intended to prevent the guide pin 17 from accidentally crossing over from the 3rd section 14c into the fourth section 14d. The engagement of the guide pin 17 in slide 14 precisely defines the movement of the cutting part 22 relative to the housing 11 and, in the illustrated embodiment, comprises two rotary movements in the 1st section 14a and in the 3rd section 14c, as well as two axial movements in the 2nd section 14b and in the 4th section 14d.

In order to ensure reliable guidance, a similar slide guide-way is preferably provided on the diametrically opposite side of the housing 11, not visible in the figures, in which a corresponding further guide pin 17 engages.

The mode of function of the test device 10 is explained in detail below. First of all, the bodily fluid or blood to be examined is introduced into the upper in-feed area 20 of the test device 10. To this end, the cap 25 is removed from the capillary tube 24 and the upper free end of the capillary tube 24 is brought into contact with a drop of blood, for example, on the finger tip of a test person. The blood enters the capillary tube 24 by capillary action of the capillary tube 24 and completely fills it. In this way, an amount of blood predefined by the volume of the capillary tube 24 can be taken up. Finally, the cap 25 with its blind hole 26 is placed on the capillary tube 24 and pushed right onto it. By this action, the volume between the base of the blind hole 26 and the upper end of the capillary tube 24 is reduced, which causes the pressure to rise and therefore the blood to be discharged from the lower end of the capillary tube 24 into an interior space 29 of the tube-shaped projection 21b of the cutting part 22.

Putting on the cap 25 causes the projection 27a of the guide part 27 of the cap 25 to engage with the retainer 22a of the cutting part 22, enabling transfer of any rotational movement of the cap 25 to the cutting part 22.

The user rotates the cap 25, which also causes the cutting part 22 to rotate as far as the guide pin 17 attached to it is able to move in the 1st section 14a of the slide 14. Subsequently, the user applies pressure to the cap 25 from above, which also causes the cutting part 22 to move downward in the axial direction of the housing and the guide pin 17 moves along the 2nd section of the slide 14. This movement of the cutting part 22 in the axial direction of the housing 11 is limited by the length of the 2nd section 14b of the slide 14. This axial movement of the cutting part 22 causes the cutting knife 23 constituted at its lower end to press against and destroy the upper covering film 32 of the upper cartridge 31. The blood located in the interior space 29 of the tube-shaped projection 21b can thus mingle with the reagent located in the cartridge 31 and react with it. The further cartridges 34 and 37 remain closed.

In order to initiate a further phase of the test, the user again rotates the cap 25 causing the guide pin 17 to travel along the 3rd section 14c of the slide 14 and enter the 4th section 14d of the slide 14 that extends in the longitudinal direction of the housing 11. In this position, it is possible for the user to press the cap 25 still further into the housing 11, which also moves the cutting part 23 within the housing 11 and pierces and destroys both the lower covering film 33 of the 1st cartridge, the adjacent upper covering film 35 of the 2nd cartridge 34, the lower covering film 36 of the 2nd cartridge 34, the adjacent upper covering film 38 of the 3rd cartridge 37 and also the lower covering film 39 of the lower 3rd cartridge 37. In this way, the blood also comes into contact with the reagents or antibodies or other reaction agents contained in the 2nd cartridge 34 and the 3rd cartridge 37 and reacts with them. The blood then enters the bowl-shaped separating element 41 from above and flows through the through-hole 42 into the strip-shaped indication element 45 located directly below it, where a change in color can occur, which the user can view from outside the housing through a window 18 (see FIG. 1).

If individual pieces of film become detached when the covering films 32, 33, 35, 36, 38 and 39 are pierced, they also fall into the bowl-shaped separating element 41 and come to rest on the spacer elements 43, so that the blocking of the through-bore holes 42 by these film pieces is prevented.

1-16. (canceled)

17. A test device for a bodily fluid of a human or animal, the test device comprising:
a tube-shaped housing;
a structure defining an in-feed area for feeding-in a prescribed quantity of the bodily fluid to be tested;
a structure defining a reaction area connected downstream of said in-feed area;
a closed separating wall disposed between and separating said in-feed area from said reaction area;
a structure defining an indication area connected downstream of said reaction area, said indication area structured and dimensioned for acceptance of an indication element;
a separating element having a defined through-hole, said separating element disposed between said reaction area and said indication area; and
a cutting device disposed, structured and dimensioned for destroying said separating wall, wherein, after destruction of said separating wall, the bodily fluid is brought into chemical reaction with at least one reagent in said reaction area and, after reacting with the reagent, the bodily fluid is passed through said through-hole into said indication area.

18. The test device of claim 17, wherein said in-feed area has a capillary tube into which the bodily fluid to be tested can be introduced by capillary action.

19. The test device of claim 18, further comprising a cap cooperating with said capillary tube, wherein the bodily fluid located in said capillary tube is thereby pressed out said capillary tube.

20. The test device of claim 19, wherein said cutting device has a cutting part which is positioned in said housing for rotational and/or axial motion and which has a cutting knife.

21. The test device of claim 20, wherein motion of said cutting part is controlled by a slide that is constituted in said housing.

22. The test device of claim 20, wherein said cutting part engages said cap in positive connection, wherein movement of said cap causes movement of said cutting part.

23. The test device of claim 17, wherein the reagent is disposed in a cartridge, which is inserted into said housing and has at least one covering film, which can be destroyed with said cutting device.

24. The test device of claim 23, wherein said cartridge has a tube-shaped housing part, which is inserted into said housing so that it fits tightly, and which is closed with said covering film at both ends thereof in a direction of said housing.

25. The test device of claim 23, wherein multiple cartridges are disposed one behind an other in an axial direction of said housing.

26. The test device of claim 25, wherein said cartridges are pressed against each other using a clamping element.

27. The test device of claim 17, wherein said separating element is bowl-shaped and fits tightly in said housing.

28. The test device of claim 27, wherein said through-hole is constituted in said separating element.

29. The test device of claim 17, wherein said through-hole is surrounded by spacer elements.

30. The test device of claim 17, wherein a retainer for the indication element is constituted on a side of said separating element facing said indication area.

31. The test device of claim 17, wherein the indication element is disposed directly at a mouth of said through-hole.

32. The test device of claim 17, wherein a holder engages said indication element at an end thereof facing away from said separating element.
